(19) United States
(12) Patent Application Publication
(10) Pub. No.: US 2007/0016121 A1
(43) Pub. Date: Jan. 18, 2007

(54) An orthopedic appliance and method to reduce anterior dislocation of shoulder and to provide post reduction immobilization

(76) Inventors: Marek Kaminski, Fall Creek, WI (US); Zofia Stanislawa Kaminski, Fall Creek, WI (US)

Correspondence Address: Marek Kaminski, 4451 Dewitz Rd., Fall Creek, WI, 54742, US

(21) Appl. No.: 11/160,890

(22) Filed: Jul. 14, 2005

(51) Int. Cl. A61F 5/00

(52) U.S. Cl. 602/20

(57) ABSTRACT

The disclosure is of an orthopedic appliance designed to facilitate the reduction of anterior dislocation of person's shoulder by utilizing the principle of a third class lever. After the elbow is immobilized against chest wall with a chest belt, the reduction is achieved by inflating a pouch tightly secured with straps under the involved axilla. Once the reduction is accomplished, immobilization of the injured shoulder is provided by transforming the chest belt into a waist belt to which an elbow cuff and a sling are anchored. This injury-specific immobilization provides for limited mobility and usage of the forearm, wrist, and hand.

### BACKGROUND

This invention relates to an orthopedic appliance designed to facilitate the reduction of anterior dislocation (subcoracoid dislocation) of person's shoulder and to provide post reduction immobilization appropriate for the injury.

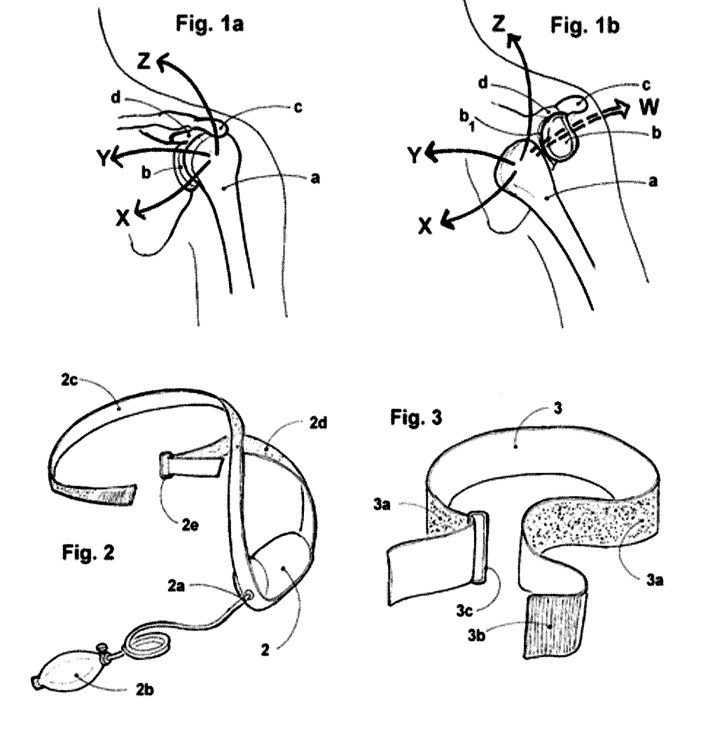

The anterior shoulder dislocation represents the most common dislocation of a major joint. The joint (FIG. 1a) consists of the head of the humerus (a) moving against a component of shoulder blade called the glenoid (b). The upper rim of the joint is formed by two bony finger-like formations: the acrominon (c) and the coracoid (d). However, no bony structure protects the humerus from slipping forward and/or down. Instead, it is held in place by the powerful, fine tuned shoulder muscle groups pulling the humerus in directions X, Y and Z (solid arrows). As a result of trauma or inopportune motion, the head of the humerus slips, most often forward and down (FIG. 1b). The same powerful muscle groups that normally keep it in place against the glenoid (b) now pull it towards the centerline of the body. Consequently the humerus slips further and gets caught behind a rather prominent rim of the glenoid (b₁). Progressive spasms of the shoulder muscles, pulling in directions X, Y and Z hold the humerus firmly in this new position which is called anterior dislocation. The treatment of this injury is called reduction and is preformed manually. However, the most direct approach, i.e. pushing the head of the humerus in the direction W (FIG. 1b, dotted arrow), is not possible. The operators' fingers placed in the arm pit (there is no room for anything else) do not provide enough strength to counteract spasm of the powerful shoulder muscles. Therefore, treatment of this injury often requires often heavy chemical sedation and muscle relaxation. The reduction itself is accomplished by various maneuvers like pulling or rotating the injured shoulder. Medical texts recommend a multiplicity of maneuvers designed to indirectly overcome the force of the muscles pulling the humerus in. Those maneuvers require the application of a significant indirect force that can easily aggravate the existing or cause new tension injuries to the already compromised ligaments, muscles, tendons, nerves, and blood vessels. This risk is particularly high in the elderly, who represent a significant percentage of the injured.

The only instance of the previous art related to the reduction of shoulder dislocation pertains to the Stimson Technique. This technique, often ineffective, involves laying a person prone, face down, on the edge of gurney with the involved arm hanging down. In the original description of the maneuver, the force pulling arm down is created by hanging a weight (a water filled bucket) on patient's wrist with an improvised wrist strap made of—for example—a bandage. Watkins et. al. (U.S. Pat. No. 5,997,494) proposed a forearm strap with loops in which weighted units could be placed. Thus, the art represents an incremental improvement of an established technique; rather than a new approach to the problem itself.

After the reduction is accomplished, the injured shoulder is traditionally placed in a shoulder immobilizer for several weeks. This is done in order to prevent a reoccurrence of the dislocation. However, the data regarding the benefit of this practice remains controversial. Some studies show a reduction of dislocation reoccurrence but others do not (Other publications: 1, 2). At the same time, there is no controversy with regard to the inconvenience of wearing a cumbersome and constricting immobilizer. Several shoulder immobilizing devices were proposed. For example devices proposed by Florek (U.S. Pat. No. 4,480,637, Marino (U.S. Pat. No. 4,751,923), Marble (U.S. Pat. No. 5,095,894), Brukhead (U.S. Pat. No. 5,334,1325) and Johnson (U.S. Pat. No. 5,358,470) exhibit a high level of complexity aimed at achieving total and complete immobilization. This might be advantageous for other types of injuries, however, in post dislocation recovery it is clearly excessive. Furthermore, the listed devices immobilize arm against the anterior chest. This type of immobilization places the weight of the arm anteriorly and medially of its natural position. As a result, the center of gravity of the upper body is shifted forward and towards the midline of the body, disturbing the natural balance of person's walk.

### OBJECTS OF THE INVENTION

The a primary objective of the present invention is to overcome disadvantages and problems relative to the inability of the present techniques to directly counteract the muscles holding the joint out of place and thus having to resort to indirect, forceful manual manipulation.

Since the indirect manual reduction requires forceful manipulation of an already injured joint, another objective of the present invention is to minimize the risk of additional tension injuries to the already compromised nerves, blood vessels, and tendons during the dislocation reduction procedure.

Because the indirect forceful manual reduction is extremely painful, a significant level of often risky and costly sedation and anesthesia is required during the procedure. Therefore, another objective of the present invention is to reduce anesthesia and sedation related risks and costs.

These objectives are achieved by a mechanical means utilizing the principle of a third class lever, with a force delivered by means of gentle pneumatic pressure directly counteracting the spasm of the shoulder muscles.

Immediately after the reduction, the patient is usually at least somewhat sedated and may inadvertently move the shoulder in a direction and manner causing an additional damage or even a recurrence of the dislocation. Therefore, yet another objective of this invention is to prevent this unfavorable occurrence by providing continuous immobilization during and immediately after the procedure.

Another objective of the present invention is directed towards overcoming the disadvantages and problems relative to rigid and constricting post-reduction immobilization of the shoulder, the elbow, wrist, and hand proposed in the previous art by Florek (U.S. Pat. No. 4,480,637, Marino (U.S. Pat. No. 4,751,923) and Marble (U.S. Pat. No. 5,095,894). This level of splinting in post reduction recovery is not only excessive and unduly uncomfortable but also can adversely affect the outcome, as some authorities advocate favorable effects of allowing for gradual progress in the elbow, wrist, and hand mobility during the recovery (Other publications: 3). This objective is accomplished by a selective and adjustable immobilization of the elbow with a cuff and supporting the wrist with a sling. Adjustability of both the elbow cuff and the sling allows gradual reducing of the tension and permits hand and shoulder use in accordance with the progress of the recovery. Yet another objective of this invention is directed towards overcoming the disadvantages of the present shoulder immobilization techniques that place the injured arm, wrist and hand against the anterior chest and, thus, disturb the natural balance of the upper body by shifting its gravity center towards the uninvolved side.

This objective is accomplished by immobilizing the elbow against the lateral rather than the anterior chest and by leaving the forearm parallel rather than perpendicular to the saggital (anterior-posterior) plane of the body.

### PREFERRED EMBODIMENT

The invention is comprised of an inflatable auxiliary pouch with straps (FIG. 2) and a three part elbow/shoulder immobilizer. (FIGS. 3, 4, 5)

The inflatable cylindrical pouch (FIG. 2) is made of a strong airtight material (2). It is inflated through rubber or plastic tubing located at front of the pouch (2a). The tubing is connected to a pump (bulb) allowing for the gradual inflation of the pouch (2b). The pump is equipped with two valves. The inlet “one way” valve allows air into bulb only. The outlet “two way” valve directs the air to the pouch during inflation, allowing for gradual pressure built up. At the same time, if needed, it permits immediate or gradual release/adjustment of the pressure. In an alternative embodiment, instead of a pump, a connector is provided to connect the tubing with the pumps that are used to inflate the cuffs of blood pressure sphingometers that are widely available in health care facilities.

The pouch is attached to the strap at the bottom. The ends of both straps (2c, 2d) are equipped with velcro locks. The shorter back strap (2d) has a buckle (2e). The front strap (2c) is long enough to be placed over the shoulder, across the back of the neck, and under the opposite shoulder.

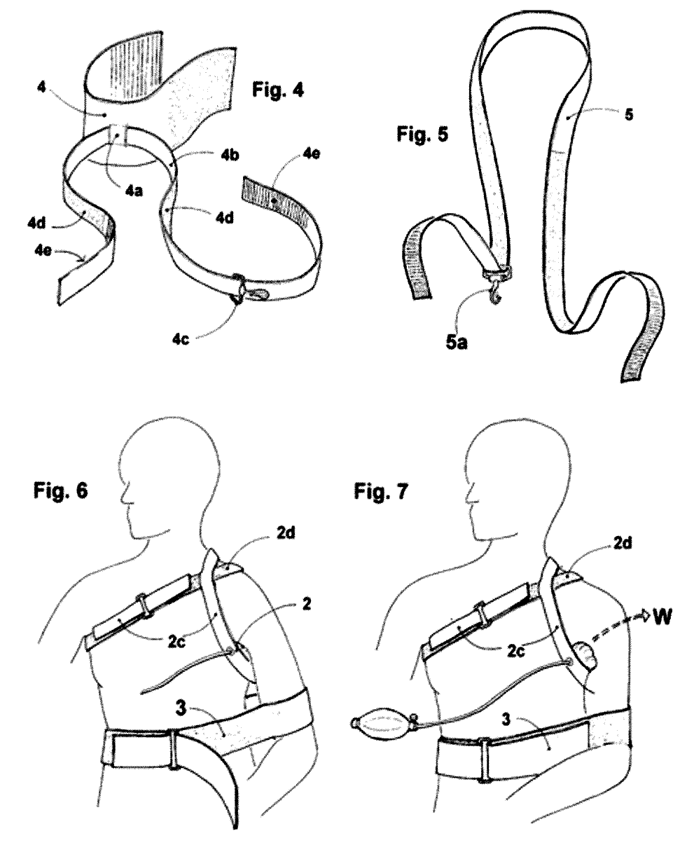

The elbow/shoulder immobilizer consists of a chest belt (FIG. 3), an elbow cuff with straps (FIG. 4), and a sling (FIG. 5).

The chest belt (3) (FIG. 3), is made of a strong fabric. The entire outer side of the belt is covered with non-engaging velcro (3a), except for the ends equipped with the engaging velcro strips (3b). The belt is also provided with a rectangle buckle (3c) which allows for unlimited adjustment of the belt's length.

The elbow cuff (4) (FIG. 4), is made of a stiff fabric and is supplied with velcro on both ends so that its length can accommodate any circumference of a distal arm. In the center of the cuff there is a double vertical slit (4a) through which a strap (4b) is threaded. The strap is made of a soft but strong fabric about an inch wide. Its outside surface is covered with non-engaging velcro (4d) while there are engaging velcro strips provided on both ends (4e). The anterior (front) end of the strap is provided with a snap buckle held by a velcro lock for length adjustment (4c). Once the strap is applied, the hook snaps on the chest belt buckle (3c) and the posterior (back) end of the strap (4e) attaches to the non-engaging velcro (3a) on the chest belt. Alternatively, the strap can be attached to a regular dress belt. In the front, a snap buckle is hooked to the belt's buckle. On the back the strap is fixed to the belt with a loop closed with velcro.

The sling (FIG. 5) is designed to support the forearm in position, allowing immobilization of the humerus in its natural resting position along the long axis of the chest. Again, both ends of the sling (5) are provided with velcro locks for length adjustment. The front end of the sling is provided with a snap buckle (5a) which hooks to the chest belt buckle (3c). Alternatively, the chest belt buckle could be wide enough for the sling strap to pass through. On the opposite end, velcro allows for a wrist loop for be made at the most comfortable length and diameter.

Method

The reduction of the dislocation and the consequent immobilization of the shoulder is accomplished in 4 simple steps.

Step 1

Once the injured patient is placed in a comfortable, semi-reclined position, the length of the chest belt is adjusted to exceed the estimated combined circumferences of patient's elbow and waist (FIG. 6). Next, the belt (3) is placed loosely around the injured arm and chest and the completely deflated pouch (2) is gently but firmly secured under the injured joint with the straps. The back strap (2d) is placed over the shoulder and its length adjusted so that the buckle rests in the center of the chest. The front strap (2c) is also placed over the injured shoulder crossing the back strap, across the back of the neck and under the uninvolved shoulder. The loop is closed by threading it through of the back strap buckle. Once the straps are properly positioned they are tightened on both ends using velcro. Crossing the straps over the shoulder secures the pouch in the most desirable position i.e. next to the dislocated head of the humerus.

Step 2

Once the deflated pouch is firmly secured under the patient's axilla, the elbow is gently directed towards the chest by gradually pulling the free end of the chest belt through the buckle until the elbow is tightly splinted against the chest (FIG. 7).

The immobilized elbow provides the fulcrum to the third class lever formed by the humerus. The force is delivered by inflating the pouch placed practically next to the load consisting of the dislocated head of the humerus. Thus, a very favorable force multiplication is achieved in the most desired direction W, i.e. directly counteracting the spasm of shoulder muscles.

Next the pouch is gradually inflated. Once the circumference of the head of the humerus passes over the rim of the glenoid (b₁) (FIG. 1b), shoulder muscles pull it back into its normal position (FIG. 1a) and the reduction of the dislocation is concluded. The reduction could be facilitated by a gentle external rotation of the flexed elbow. While the shoulder remains immobilized by a chest belt, the pouch is rapidly deflated and, after loosening of the chest belt, removed.

Step 3

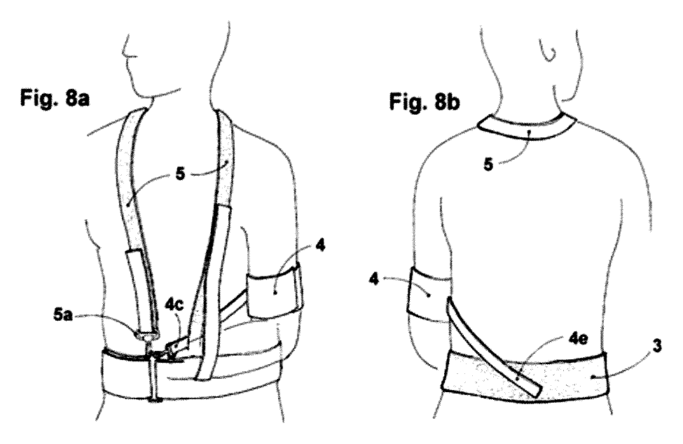

Next, the loose belt is lowered and buckled again, but this time at patient's waist level, where belts are usually placed and where they feel most comfortable (FIG. 8A). This represents a significant improvement over the previously proposed immobilizers that squeeze the patients at the level of upper abdomen and lower chest: Garnett (U.S. Pat. No. 3,780,729), Florek (U.S. Pat. No. 4,480,637), Marino (U.S. Pat. No. 4,751,923), Marble (U.S. Pat. No. 5,095,894), Johnson (U.S. Pat. No. 5,358,470).

Then the sling strap (5) is hooked (5a) to the belt buckle, crossed around the back of the neck, and closed over the patient's wrist on the injured side. Its length is then adjusted at both ends by means of velcro. Subsequently, the elbow cuff (4) is positioned loosely above the elbow that is already supported with a sling. First, the front end of the strap is hooked to the belt buckle (4c) (FIG. 8a). Then, the back end of the strap (4e) is velcro-attached to the posterior surface of the chest belt (3) (FIG. 8b). Once the shoulder finds its most natural and comfortable position by moving elbow cuff (4) along the strap, the strap is tightened on both ends, immobilizing the elbow in the chosen position.

Thus, the shoulder joint is immobilized in the most natural position, i.e the head of the humerus placed directly against the center of the glenoid. The humerus is also maintained in its natural resting position i.e. along the saggital plane of the upper chest. The flexed forearm is also kept naturally i.e. parallel to saggital (anterior-posterior) plane of the upper chest. This is a significant improvement over the previous art where the forearm is splinted against anterior chest, and thus the humerus is pulled forward and internally rotated: (U.S. Pat. No. 3,780,729), Hubbard at. al. (U.S. Pat. No. 4,372,301), Florek (U.S. Pat. No. 4,480,637), Marino (U.S. Pat. No. 4,751,923), Marble (U.S. Pat. No. 5,095,894) and Johnson (U.S. Pat. No. 5,358,470). This kind of anterior splinting not only places humerus in unnatural position but also disturbs the natural balance of the upper body by shifting its gravity center towards the uninvolved side.

In the present invention, the sling allows for maintaining a certain degree of motion in the elbow joint as well as limited usage of the wrist.

Furthermore, the main belt placed at the most natural position, i.e. at the waist rather than squeezing patient's chest. This is not only more comfortable but, it allows for elbow immobilization by anchoring it to a regular dress belt.

Furthermore, the present design of elbow straps allowing the creation of a pulling tension on the humerus could be useful in treatment of the fracture of that bone.

What is claimed is:

1. Apparatus for reducing anterior dislocation of the shoulder that utilizes the principle of a third class lever where fulcrum is achieved by immobilization of the elbow against chest wall and the force delivered by exerting pneumatic pressure next to the load consisting of the dislocated head of the humerus.

2. Apparatus according to the claim 1 wherein the pneumatic pressure is delivered by gradual inflation of a pouch placed directly under the dislocated joint.

3. Apparatus according to claim 2, wherein the pouch is made of an airtight material and is connected with a tube to a manual or mechanical air pump, allowing for gradual inflation of the pouch while maintaining the pressure and immediate release of the pressure when needed.

4. Apparatus according to claim 2, wherein instead of a separate pump, a connector is provided allowing for inflation of the pouch with standard pumps used in medical facilities for inflating sphingometr cuffs.

5. Apparatus according to claim 2 wherein the said pouch is attached by its bottom to a strap allowing for firm placement of the pouch under the injured arm,
said strap being made of a strong fabric with both ends provided with velcro locks,
said strap being divided into to uneven parts by the pouch: the front portion being longer than the back,
said strap being of a sufficient length to form a loop that crosses over the injured shoulder, over the back of the neck and under the opposite shoulder,
the back end of said strap provided with a rectangle buckle for length adjustment and for closing the loop on the front of the chest.

6. Apparatus according to claim 1 wherein the elbow is splinted by means of an adjustable chest belt.

7. Chest belt according to claim 6 made of a strong fabric and the length exceeding circumference of patient's chest and elbow,
said belt having its outer surface covered with non-engaging velcro and with velcro engaging strips on both ends,
said belt being provided with a rectangular buckle allowing for dual adjustment of the length.

8. Chest belt according to claim 6 that once the dislocation is reduced, is repositioned to the waist level and thus transformed into a waist belt that serves as an anchor for post-reduction shoulder immobilizer.

9. Shoulder immobilizer according to claim 8 consisting of an elbow immobilizer and a sling.

10. Elbow immobilizer according to claim 9 consisting of an elbow cuff and an adjustable strap.

11. Elbow cuff according to claim 10 made of a strong stiff fabric and provided with velcro locks,
said cuff being provided with two slits for the elbow strap to be threaded through,
said slits being located in the center of the cuff, being perpendicular to the long axis of the cuff and of the length slightly exceeding the width of the elbow strap.

12. Strap according to claim 10 made of a soft but strong fabric tape,
the external surface of the said strap being covered with non-engaging velcro and engaging velcro strips provided on both ends,
said strap being provided with a snap buckle on the front end for hooking to the buckle of the waist belt,
said strap being threaded though a double slit of the elbow cuff and its back end attached to the back of the waist belt with a velcro lock.

13. Sling according to claim 9 being made of a strong fabric tape and supplied with velcro locks on both ends,
said sling being provided with snap buckle on one end and vlecro adjusted wrist loop on the other end,
once the dislocation is reduced, said sling is hooked to the waist belt buckle and its free end crossed over the back of the neck in the direction from an uninjured towards an injured extremity and the wrist placed in the velcro wrist loop.

14. Method of treating (reducing) an anterior dislocation of shoulder joint that utilizes the principle of third class lever where fulcrum is achieved by the immobilization of elbow against chest wall and a force delivered by exerting pneumatic or hydraulic pressure next to the load consisting of the dislocated head of the humerus.

15. Method of treating (reducing) an anterior dislocation of shoulder joint according to claim 14 wherein the pressure is delivered by securing an inflatable pouch under the injured shoulder and gradually inflating the pouch.

16. Method of treating (reducing) an anterior dislocation of shoulder joint according to claim 14 wherein the distal humerus/elbow is immobilized by being splinted against lateral chest by means of adjustable chest belt.
